(19) United States
(12) Patent Application Publication
(10) Pub. No.: US 2016/0135996 A1
(43) Pub. Date: May 19, 2016

(54) Earamps

(71) Applicants: Justin Christopher Lee, Mary Esther, FL (US); Kenneth W. Tissot, Mary Esther, FL (US); Jonathan L. Ritter, DeFuniak Springs, FL (US)

(72) Inventors: Justin Christopher Lee, Mary Esther, FL (US); Kenneth W. Tissot, Mary Esther, FL (US); Jonathan L. Ritter, DeFuniak Springs, FL (US)

(73) Assignees: Justin Christopher Lee, Mary Esther, FL (US); Kenneth W. tissot, Mary Esther, FL (US); Jonathan L. Ritter, DeFuniak Springs, FL (US)

(21) Appl. No.: 14/756,115

(22) Filed: Sep. 11, 2015

Related U.S. Application Data
(60) Provisional application No. 62/123,468, filed on Nov. 19, 2014.

(51) Int. Cl. A61F 11/00

(52) U.S. Cl. CPC A61F 11/008

(57) ABSTRACT

A device with an adjustable horizontally faced adjustable headband, including recyclable plastic elements of and adapter that attaches multiple spherical cups. Attached to the spherical cups are styrofoam liners, which provide comfort and solidify sounds within the ear canal for a personal amphitheater experience.

### FIELD

The present application relates to a mechanical device that is modular in design with a retractable headband attached to spherical cups through an adapter assembly that face outward to provide an enhanced sound experience.

### BACKGROUND

Hearing devices have become a billion dollar industry around the world as a means of improving hearing for the elderly, disabled, hearing impaired, children in overcrowded classrooms and for entertainment purposes. With the increase of costs for such devices along with the economies fiscal fluctuation, it becomes difficult to afford practical and beneficial devices. It's not fair for the average consumer to suffer professionally and academically due to the lack of funds for a product that should be affordable. Along with the high prices tags, technology is shifting towards green friendly devices that are made from recyclable plastics that help preserve the natural environment. These materials are inexpensive, child friendly, and preserve the environment.

### SUMMARY

By providing a device with a retractable headband attached to recyclable plastic spherical assemblies costs are reduced while the ability to focus enhanced sound is significantly increased.

The disclosed exemplary embodiments provide a device which is primary retractable and spherical.

In one embodiment the headband entails a plastic retractable unit that is designed for all ages. The adjustable headband is placed to the rear of an individual's head facing inward for comfort and modernization. The lightweight plastic is durable which allow for simple attachment over the ear for an enhanced listening experience.

In an embodiment attached to the headband are two spherical assemblies that fit all ages and are made from recyclable plastics. The assemblies are designed as personal amphitheaters.

Further objects, features, advantages and properties of device according to the present application will become apparent from the detailed description.

### BRIEF DESCRIPTION OF THE DRAWINGS

In the following detailed portion of the present description, the teachings of the present application will be explained in more detail with reference to the example embodiment shown in the drawings, in which:

FIG. 1 provides a front and rear view of the Earamps assembly. The assembly is comprised of component label 200 the headband, component label 201 the adapter plate, component label 202 the 4″ spherical cups, and component label 203 the Styrofoam tubing. The FIG. 1 assembly is made from environmentally friendly plastic and is also lightweight. The assembly is placed behind the head and sits behind both ears in a horizontal position.

FIG. 2 provides a front and side view of the circular 10″ headband. The headband is lightweight, environmentally friendly plastic, and adjustable to accommodate youths and adults. The FIG. 2 curved embodiment is placed to the rear of the head in a horizontal position. FIG. 2 also provides connecting insertions on the inner portion of the headband, which allows for assembly to the FIG. 3 spherical cups through the FIG. 4 adapter plates.

FIG. 3 provides a side and top view of the spherical ear cups, which are made to enhance the sound as a main component for the Earamps invention. The spherical cups are made from environmentally friendly plastic and are made in multiples of two for each set of Earamps. There is a ⅜″ hole in the center of FIG. 3 which allow for connection to FIG. 2 through the adapter plate in FIG. 4. The FIG. 3 spherical ear cups are placed along the side of the head around the ear, open faced forward.

FIG. 4 provides a front and side view the adapter plate, which is made from environmentally friendly plastic. FIG. 4 has an insertion that is initially placed through the hole of FIG. 3 and is then connected to FIG. 2. FIG. 4 is lightweight and child friendly.

FIG. 5. provides a front and side view of the Styrofoam tubing, which is placed along the edge of FIG. 3 spherical cups. Once assembled along the edge of FIG. 3, FIG. 5 lies along the head of the user and provides comfort and ease. FIG. 5. is lightweight and child friendly.

### DETAILED DESCRIPTION

In the following detailed description, the device according to the teachings for this application in the form of a personal amphitheater to assist with hearing amplification will be described by the embodiments. It should be noted that the improved hearing device is environmentally friendly while catering to the needs of individuals all ages, young and old. The affordable amplifiers are designed to provide comfort that not only amplifies the volume of sound, but also amplifies the quality of sound. The device was designed as an affordable opportunity for children to learn effectively and better understand teachers in overcrowded classroom situations, there are also a number of other applications that can have relevance including medical, entertainment, domestic, and professional environments.

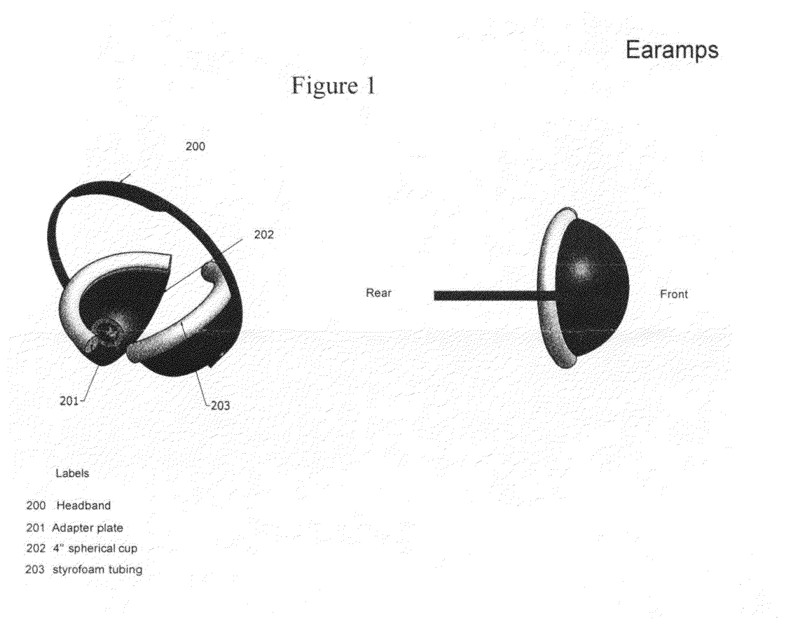

The component in FIG. 1, provides an illustration of all invention components including he; label 200 headband, label 201 adapter plate, label 202 4″ spherical cups, and label 203 Styrofoam tubing.

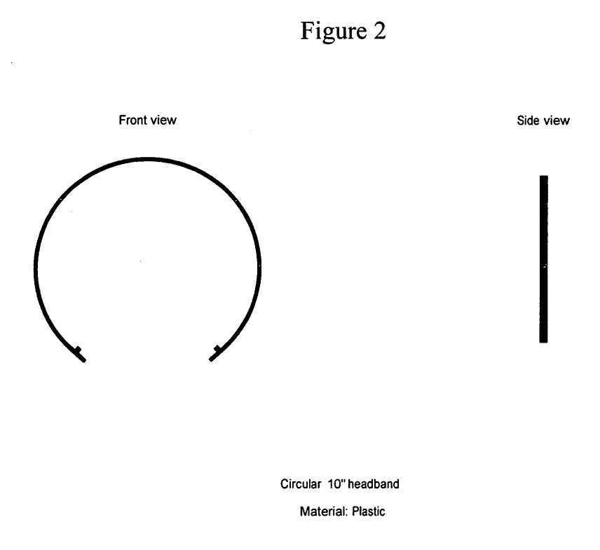

The component in FIG. 2, is a 10″ headband, which is made from close cell plastic to provide pressure against the back of the head and rest behind both ears. The headband is positioned horizontally as it faces openly in the forward position. The embodiment is made of recyclable plastic and is adjustable for fitting all ages. The FIG. 2 can be adjusted accordingly to fit children and adults.

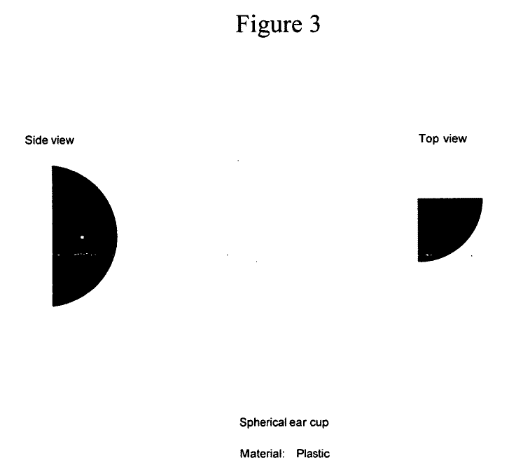

The component in FIG. 3, is a 4″ spherical cup that is designed as a multiple of two which is placed over the ears with a ⅜″ thru hole cut into for attachment to the first embodiment through the third embodiment. The spherical cups are also assembled with an open face pointing in the forward direction. The cups are also made from recyclable plastic, these uniquely designed cups function as the hearing amplifiers to focus vibration waves directly to the external ear canal for an enhanced effect.

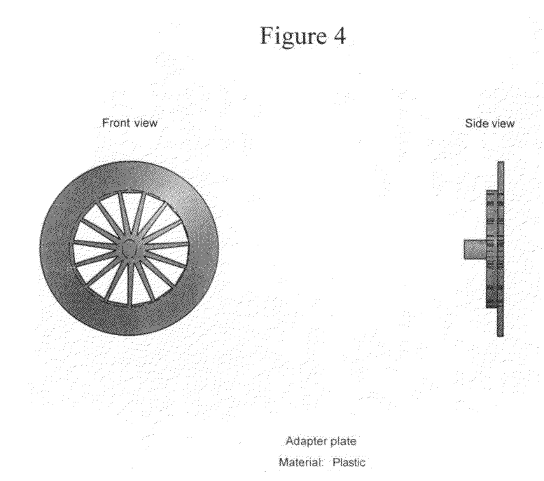

The component in FIG. 4, is the adapter plate for the purpose of connecting FIG. 2 and FIG. 3. The adapter plate, FIG. 4, is connected through FIG. 3 and to FIG. 2. The FIG. 4 is ½″ is diameter and is made from recyclable plastic. This units solely function is to maintain the assembly of the FIG. 2 and FIG. 3.

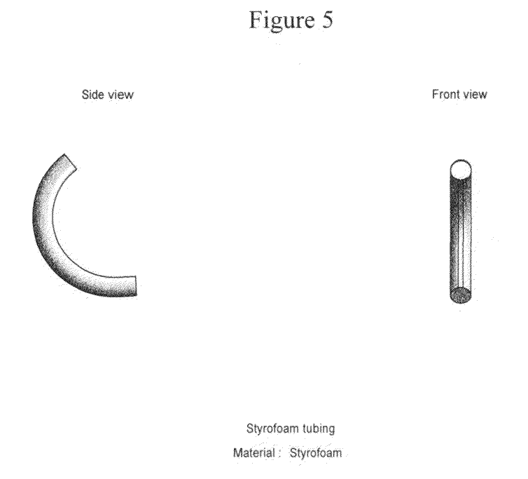

The component in FIG. 5 is the 4″ long styrofoam tubing, which is tracked around the liner on a single plane of FIG. 3 for the purpose of comfort. FIG. 5 is attached to both of the spherical cups, FIG. 3, to increase comfort for the users. The material of FIG. 5 has a small curvature for the intended use of simple integration.

1. An environmentally plastic adjustable headband front faced horizontally has a curved shape for displacement towards the back of the head to rest on the ears.

2. A device according to claim 1, wherein the front face of two spherical cups are attached to an adapter to provide stability.

3. A device according to claim 2, wherein the adapter is then connected thru a hole of the two spherical cups to the headband that are opened face pointing forward and enclosed around the ear.

4. A device according to claim 3, wherein there is a Styrofoam liner that trails along the edges of the spherical cups.
